(19) United States
(12) Patent Application Publication
(10) Pub. No.: US 2026/0355208 A1
(43) Pub. Date: Nov. 12, 2026

(54) COMBINATIONS OF KRAS G12C INHIBITOR, EGFR INHIBITOR AND SOS1 INHIBITOR

(71) Applicant: MIRATI THERAPEUTICS, INC., PRINCETON, NJ (US)

(72) Inventor: LINA DING, SUDBURY, MA (US)

(73) Assignee: MIRATI THERAPEUTICS, INC., PRINCETON, NJ (US)

(21) Appl. No.: 19/665,058

(22) Filed: May 1, 2026

Related U.S. Application Data
(60) Provisional application No. 63/798,700, filed on May 2, 2025.

(51) Int. Cl. A61K 31/5377; A61K 31/519; A61P 35/00

(52) U.S. Cl. CPC A61K 31/5377; A61K 31/519; A61P 35/00

(57) ABSTRACT

The present disclosure provides combinations comprising (i) Compound A:

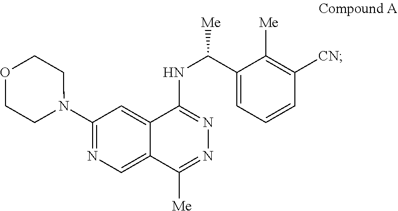 [SMILES: Cc1c(C#N)cccc1[C@@H](C)Nc1nnc(C)c2cnc(N3CCOCC3)cc12]

or a pharmaceutically acceptable salt thereof, (ii) a KRAS G12C inhibitor and, (iii) an EGFR inhibitor.

### CROSS REFERENCE TO RELATED APPLICATIONS

This application claims the benefit of U.S. Provisional Application No. 63/798,700, filed May 2, 2025, the entire content of which is hereby incorporated herein by reference.

### FIELD

The present disclosure provides combinations of a KRAS inhibitor, an EGFR inhibitor and a SOS1 inhibitor. Methods of treating cancers, comprising administering the combinations are also provided.

### BACKGROUND

The KRAS oncogene is a member of the RAS family of GTPases that are involved in numerous cellular signaling processes. KRAS mutations are gain-of-function mutations that are present in up to 30% of all tumors, including as many as 90% of pancreatic cancers. Single nucleotide substitutions that result in missense mutations at codons 12 and 13 of the KRAS primary amino acid sequence comprise approximately 40% of KRAS driver mutations in lung adenocarcinoma, with a G12C transversion being the most common activating mutation. KRAS G12C mutations occur in about 13% of lung adenocarcinomas and about 3% of colorectal adenocarcinomas and are also present in cancers of the breast, bladder, cervix, ovaries, pancreas and uterus. KRAS G12D mutations occur in 40% of all pancreatic ductal adenocarcinoma patients, 13% of all colorectal carcinoma patients, 4% of all non-small cell lung carcinoma patients and 3% of all gastric carcinoma patients. See, for example, Lee, J. K., Sivakumar, S., Schrock, A. B. et al. Comprehensive pan-cancer genomic landscape of KRAS altered cancers and real-world outcomes in solid tumors. npj Precis. Onc. 6, 91 (2022). https://doi.org/10.1038/s41698-022-00334-z. Due to the clinical significance of this protein, many attempts have been made to develop KRAS inhibitors, but such attempts have been mostly unsuccessful. Accordingly, agents that inhibit mutant KRAS are desired.

SOS proteins (including SOS1 and SOS2) bind to RAS through a binding site that promotes nucleotide exchange as well as through an allosteric site that binds GTP-bound RAS-family proteins and increases the function of SOS. Compounds that block the interaction of SOS1 and RAS-family members can prevent the recycling of KRAS into the active form and may provide a therapeutic benefit for a wide range of cancers.

The epidermal growth factor receptor (EGFR) gene, encodes a 170-kDa transmembrane tyrosine kinase receptor. The EGFR is activated by binding to its ligands such as epidermal growth factor or transforming growth factor-alpha, resulting in homodimerization or heterodimerization with another member of the EGFR family. Gene amplification and/or protein overexpression of EGFR have been observed in a variety of solid tumors, including lung, colorectal, urinary bladder, breast, head, neck, esophageal and gastric carcinomas. In some tumors such as non-small cell lung carcinoma and colorectal carcinoma, increased EGFR expression is associated with advanced stage and an unfavorable prognosis.

Combining newly discovered KRAS G12C inhibitors, EGFR inhibitors with SOS1 inhibitors provides an opportunity to successfully treat cancers by simultaneously attacking multiple mechanisms of action. There remains an unmet medical need for these effective therapies, e.g., combination therapies, for the treatment of cancers.

### BRIEF SUMMARY OF THE DISCLOSURE

In one aspect, the present disclosure provides a method of treating cancer in a subject in need thereof, the method comprising administering to the subject (i) a SOS1 inhibitor (ii) a KRAS inhibitor and (iii) an EGFR inhibitor.

In some aspects, the EGFR inhibitor is selected from cetuximab, panitumumab, zalutumumab, nimotuzumab, matuzumab, erlotinib, gefitinib, afatinib, lapatinib, osimertinib, brigatinib, and icotinib, or a pharmaceutically acceptable salt thereof. In some aspects, the SOS1 inhibitor is Compound A:

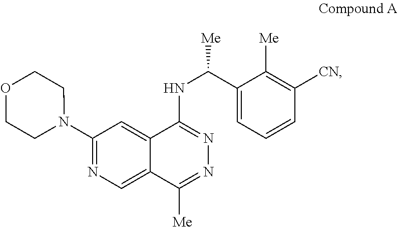 [SMILES: Cc1c(C#N)cccc1[C@@H](C)Nc1nnc(C)c2cnc(N3CCOCC3)cc12]

or a pharmaceutically acceptable salt thereof.

In some aspects, the KRAS inhibitor is a KRAS G12C inhibitor is selected from adagrasib, sotorasib, JAB-21822, GDC-6036, JDQ443, D-1553, GH35, GFH925, BPI-421286, LY3537982, RMC-6291, RMC-8839, HBI-2438, and JNJ-74699157.

In some aspects, the cancer is pancreatic cancer, colorectal cancer, lung cancer, non-small cell lung cancer, gastric cancer, breast cancer, bladder cancer, cervical cancer, ovarian cancer, cancer of the uterus, or a combination thereof. In some aspects, the cancer is pancreatic cancer, colorectal cancer, or non-small cell lung cancer.

### DETAILED DESCRIPTION

One aspect of the present disclosure is based on the use of a combination of (i) Compound A:

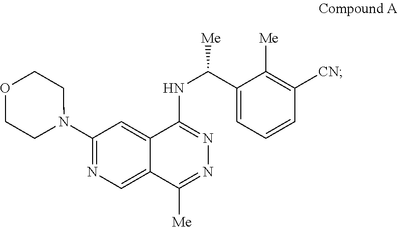 [SMILES: Cc1c(C#N)cccc1[C@@H](C)Nc1nnc(C)c2cnc(N3CCOCC3)cc12]

or a pharmaceutically acceptable salt thereof, (ii) a KRAS G12C inhibitor and (iii) an EGFR inhibitor. In one aspect, the KRAS G12C inhibitor is selected from adagrasib, sotorasib, JAB-21822, GDC-6036, JDQ443, D-1553, GH35, GFH925, BPI-421286, LY3537982, RMC-6291, RMC-8839, HBI-2438, and JNJ-74699157, or a pharmaceutically acceptable salt thereof. In one aspect, the EGFR inhibitor is selected from cetuximab, panitumumab, zalutumumab, nimotuzumab, matuzumab, erlotinib, gefitinib, afatinib, lapatinib, osimertinib, brigatinib, and icotinib, or a pharmaceutically acceptable salt thereof.

### DEFINITIONS

All technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that embodiments described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other.

The term “about,” as used herein, includes the recited number±10%. Thus, “about 10” means 9 to 11. As is understood by one skilled in the art, reference to “about” a value or parameter herein includes (and describes) instances that are directed to that value or parameter per se. For example, description referring to “about X” includes description of “X”.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The combination therapy can provide “synergy” and prove “synergistic,” i.e., the effect achieved when the active ingredients used together is greater than the sum of the effects that results from using the compounds separately. A synergistic effect can include a significantly reduced effective dose for the combination of the two active ingredients as compared to the effective dose of each active ingredient when administered separately. A synergistic effect can also include a reduction in toxicity for the combination of the two active ingredients as compared to the toxicity of each active ingredient when administered separately. A synergistic effect can also be an effect that cannot be achieved by administration of any of the active ingredients as single agents. The synergistic effect can include, but is not limited to, an effect of treating cancer by reducing tumor size, inhibiting tumor growth, or increasing survival of the subject. The synergistic effect can also include reducing cancer cell viability, inducing cancer cell death, and inhibiting or delaying cancer cell growth. A synergistic effect can be attained, for example, when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. Administration of two or more therapeutic agents includes simultaneous (concurrent) and consecutive administration in any order.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, combinations of Compound A and chemotherapeutic agents of the present disclosure can be used in treating diseases and conditions, such as proliferative diseases, wherein a chemotherapeutic agent and/or inhibition of KRAS provides a benefit.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 20% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

The terms “individual” or “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers.

### I. Compound A

Compound A and a method for its preparation are described in U.S. Pat. No. 11,702,418.

In some aspects, Compound A is administered as a free base. In some aspects, Compound A is adminstered as a salt.

For purposes of the present disclosure, Compound A can be dosed in a therapeutically effective amount. In one embodiment, Compound A can be dosed in amounts of about 5 mg to about 3000 mg, based upon the weight of Compound A free base, from 1 to 4 times per day.

In some aspects, the KRAS G12C inhibitor is adagrasib, or a pharmaceutically acceptable salt thereof. For purposes of the present disclosure, adagrasib can be dosed in a therapeutically effective amount. In one embodiment, adagrasib can be dosed in amounts of about 5 mg to about 3000 mg, based upon the weight of adagrasib free base, from 1 to 4 times per day. In one embodiment, adagrsib can be dosed in an amount of about 200 mg, based upon the weight of free base, once, two or three times a day. In one embodiment, adagrsib can be dosed in an amount of about 400 mg, based upon the weight of free base, once, two or three times a day. In one embodiment, adagrsib can be dosed in an amount of about 600 mg, based upon the weight of free base, once, two or three times a day. In one embodiment, adagrsib can be dosed in an amount of about 600 mg, based upon the weight of free base two times a day.

In some aspect, the EGFR inhibitor is gefitinib, or a pharmaceutically acceptable salt thereof. For purposes of the present disclosure, gefitinb can be dosed in a therapeutically effective amount. In one embodiment, gefitinib can be dosed in amounts of about 5 mg to about 3000 mg, based upon the weight of gefitinib free base, from 1 to 4 times per day. In one embodiment, gefitinib can be dosed in an amount of about 50 mg, based upon the weight of free base, once, two or three times a day. In one embodiment, gefitinib can be dosed in an amount of about 100 mg, based upon the weight of free base, once, two or three times a day. In one embodiment, gefitinib can be dosed in an amount of about 200 mg, based upon the weight of free base, once, two or three times a day. In one embodiment, gefitinib can be dosed in an amount of about 250 mg, based upon the weight of free base one, two or three times a day. In one embodiment, gefitinib can be dosed in an amount of about 500 mg, based upon the weight of free base one, two or three times a day.

### II. Methods of Treatment

In some aspects, the cancer to be treated with a combination of the disclosure is a cancer that is susceptible to KRAS G12A, KRAS G12C, KRAS G12D, KRAS G12R, KRAS G12S, KRAS G12V, KRAS G13D and/or KRAS Q61H inhibition. In some aspects, the cancer expresses a KRAS mutation and/or a KRAS copy number amplification. In some aspects, the cancer expresses KRAS G13R, Q61R, A146T, A146V, A59G, G12A, G12C, G12D, G12R, G12S, G12V, G13C, G13D, Q61H, Q61K, and/or a KRAS Q61L mutation, and/or a KRAS copy number amplification. In some aspects, the cancer expresses a KRAS G12A, KRAS G12C, KRAS G12D, KRAS G12R, KRAS G12S, KRAS G12V, KRAS G13D and/or KRAS Q61H mutation. In some aspects, the cancer to be treated with a combination of the disclosure is selected from the group consisting of lung cancer, non-small cell lung cancer (NSCLC), colorectal cancer (CRC), bladder cancer, ovarian cancer, cancer of the uterus, pancreatic cancer, for example, pancreatic ductal adenocarcinoma (PDAC), gastric cancer, breast cancer, and combinations thereof. In some aspects, the cancer is non-small cell lung cancer. In some aspects, the cancer is colorectal cancer. In some aspects, the cancer is pancreatic cancer.

In some aspects, Compound A, adagrasib and gefitinib are administered sequentially. In some aspects, adagrasib is administered first, and Compound A is administered second, and gefitinib is administered third In some aspects, Compound A is administered first, adagrasib is administered second, and gefitinib is administered third. In some aspects, Compound A, adagrasib and gefitinibare administered simultaneously.

In some aspects, the combination of the present disclosure is administered in a therapeutically effective amount sufficient to produce one or more therapeutic effects selected from the group consisting of (i) reduction in tumor size, (ii) increase in tumor regression rate, and (iii) reduction or inhibition of tumor growth. In some aspects, the combination of the present disclosure is administered in a therapeutically effective amount sufficient to produce an effect of reduction in tumor size. In some aspects, the combination of the present disclosure is administered in a therapeutically effective amount sufficient to produce an effect of increase in tumor regression rate. In some aspects, the combination of the present disclosure is administered in a therapeutically effective amount sufficient to produce an effect of reduction or inhibition of tumor growth.

In some aspects, the subject is a mammal. In some aspects, the mammal is a human.

In some aspects, a combination of the disclosure is used in combination with one or more additional therapeutic agents to treat cancer.

In some aspects, provided herein are combinations of the disclosure for use as a medicament or for use in preparing a medicament, e.g., for the treatment of cancer. In some aspects, provided herein are combinations of the disclosure for use in a method for the treatment of cancer.

A pharmaceutical composition of the present disclosure can be administered to any patient that may experience the beneficial effects of a Compound of the Disclosure.

Foremost among such patients are mammals, e.g., humans and companion animals, although the disclosure is not intended to be so limited. In some aspects, the patient is a human.

### Examples

### A. Methods

### Tumor Cell Line Culture and Cell Viability Assay.

Tumor Cell Line Culture. Three human tumor cell lines harboring KRAS G12C mutations were tested. The human NSCLC NCI-H2122 and NSCLC NCI-H2030 cell line was maintained in vitro with RPMI-1640 medium (Gibco, catalog 11875093) supplemented with 10% heat inactivated fetal bovine serum (Gibco, catalog 16140071) in a 37 °C chamber with 5% CO2. The human CRC SW837 cell line was maintained in vitro with DMEM medium (Gibco, catalog 11965092) supplemented with 10% heat inactivated fetal bovine serum (Gibco, catalog 16140071) in a 37 °C chamber with 5% CO2.

2D cell viability assay. Twenty-four hours after seeding, cells were treated with Adagrasib, Gefitinib and Compound A using the Tecan D300e system with serial dilution in an 8×8 matrix, as shown in. After 5 days of incubation, 3D Cell Titer-Glo (Promega, catalog G9683) assay was performed to determine cell viability and the IC50 values, data were fitted using the dose response algorithm in GraphPad Prism 9.4.0. Synergistic effects were evaluated using Loewe model analysis by SynergyFinder (synergyfinder.org). Values ≥10 indicate synergy while values of −10 to 10 show additivity and values under −10 show no combination activity. Results are shown in and.

### Patient-Derived Organoid (PDO) Establishment, Culture, and Cell Viability Assay.

Patient-Derived Organoid (PDO) Establishment and Culture. Two PDO lines harboring KRAS G12C mutation were tested. Surgically resected tumor tissue was obtained from patient under informed consent. Colorectal Cancer (CRC) T069C, and Pancreatic Ductal Adenocarcinoma (PDAC) BMS-2353 cells were isolated through a combination of mechanical disruption and enzymatic digestion. Tumor cells taken from a patient were grown and maintained in ex vivo culture to create a three-dimensional (3D) tissue model called “Patient-Derived Organoid (PDO)”, which closely resembles the original patient tumor. Genomic DNA was isolated from tumor organoids for whole-exome sequencing to identify tumor-specific somatic mutations and confirm KRAS G12C status. CRC organoids were established, expanded, and maintained in the complete organoid culture medium with Matrigel/CULTREX. The complete CRC organoid culture medium was prepared by adding the following components to advanced DMEM/F-12: R-Spondin-1 (200 ng/ml), Noggin (100 ng/ml), Gastrin (10 nM), EGF (50 ng/ml), A83-01 (500 nM), SB202190 (1 μM), B27 supplement (1×), N-Acetylcysteine (1.25 mM), Nicotinamide (10 mM), GlutaMax (1×), HEPES (10 mM), Penicillin-Streptomycin (100 U/ml-100 μg/ml), and Primocin (100 μg/ml). PDAC organoids were established, expanded, and maintained in the complete organoid culture medium with Matrigel/CULTREX. The complete PDAC organoid culture medium was prepared by adding the following components to advanced DMEM/F-12: Wnt3a (500 ng/ml), R-Spondin-1 (100 ng/ml), Noggin (100 ng/ml), Gastrin (10 nM), EGF (50 ng/ml), FGF-10 (100 ng/ml), A83-01 (500 nM), B27 supplement (1×), N-Acetylcysteine (1 mM), Nicotinamide (10 mM), GlutaMax (1×), HEPES (10 mM), Penicillin-Streptomycin (100 U/ml-100 μg/ml), and Primocin (100 μg/ml).

3D cell viability assay. For 3D proliferation assays, 1,000 cells per well were mixed with 10% CULTREX (R&D systems, catalog 3700-100-01) and seeded into 384-well ULA plates supplemented with Organoid Growth Medium. Twenty-four hours after seeding, CRC tumor organoids T069C and BMS-2353 were treated with Adagrasib, Gefitinib and Compound A using the Tecan D300e system with serial dilution in an 8×8 matrix, as shown in. After 5 days of incubation, 3D Cell Titer-Glo (CTG, Promega, catalog G9683) assay was performed to determine cell viability and the IC50 values, data were fitted using the dose response algorithm in GraphPad Prism 9.4.0. Synergistic effects were evaluated using Loewe model analysis by SynergyFinder (synergyfinder.org). Values ≥10 indicate synergy while values of −10 to 10 show additivity and values under −10 show no combination activity. Results are shown in and.

TABLE 1
Dilution and Concentrations of Compound
A, Adagrasib and Gefitinib

| | Concentration (uM) | | |
|---|---|---|---|
| Dilution | Compound A | Adagrasib | Gefitinib |
| 1 | 10 | 2.00 | 10 |
| 2 | 2.5 | 0.5 | 3.333333 |
| 3 | 0.625 | 0.125 | 1.111111 |
| 4 | 0.15625 | 0.03125 | 0.37037 |
| 5 | 0.03906 | 0.007813 | 0.123457 |
| 6 | 0.00977 | 0.001953 | 0.041152 |
| 7 | 0.00244 | 0.000488 | 0.013717 |
| 8 | 0 | 0 | 0 |

### B. Results

KRAS G12C mutant CRC cell lines and organoids showed differential sensitivity to single agent treatment of Adagrasib, Gefitinib, and Compound A inhibition as shown in. NCI-H2030 and BMS-2353 were resistant to Adagrasib monotherapy. A combination therapy of Adagrasib and Gefitinib inhibition exerted stronger anti-tumor activity than monotherapy in NCI-H2122. The synergy score was 10.22 without the presence of Compound A. The other 4 models tested did not exhibit obviously synergistic effect with the doublet combination of Adagrasib and Gefitinib inhibition.

With the presence of Compound A, the triplet combination therapy with Adagrasib and Gefitinib exerted much greater anti-tumor activity than any doublet in all five models tested. As shown in, in SW837 and NCI-2030 cell lines, dose-dependent anti-tumor activity was observed when adding 0.6 μM to 10 μM of Compound A to the Adagrasib and Gefitinib doublet combination. Similarly, stronger and dose-dependent synergetic anti-tumor activity was observed in T069C PDO and NCI-2122 by adding 0.2 uM-10 uM and 0.039 uM-10 uM of Compound A, respectively. In BMS-2353 PDO model, which was highly resistant to all the compounds as monotherapy, triple combination synergy was demonstrated at all doses of Compound A presence.

In summary, the in vitro cellular data demonstrated the triplet combination inhibition of Adagrasib, Gefitinib and Compound A induced stronger anti-tumor response than monotherapy and doublet combinations in CRC, NSCLC and PDAC tumor models with KRAS G12C mutation.

TABLE 2
Summary of anti-tumor Activity of Compound A,
Adagrasib and Gefitinib as Monotherapy in KRAS
G12C mutated CRC Cell Lines and Organoids

| KRAS | Indi- | | CTG IC50 (uM) | | |
|---|---|---|---|---|---|
| status | cation | Model | Compound A | Adagrasib | Gefitinib |
| G12C | CRC | SW837 | >10 | 0.0056 | >10 |
| G12C | CRC | T069C | >10 | 0.0247 | 0.2718 |
| G12C | NSCLC | NCI-H2122 | >10 | 0.099 | >10 |
| G12C | NSCLC | NCI-H2030 | >10 | 2.76 | 3.62 |
| G12C | PDAC | BMS-2353 | >10 | >10 | >10 |

TABLE 3
Summary of anti-tumor Activity of Compound A,
Adagrasib and Gefitinib as Combination Therapy
in KRAS G12C mutated Cell Lines and Organoids

| | Loewe synergy score of Adagrasib and Gefitinib | | | | | | | |
|---|---|---|---|---|---|---|---|---|
| | with various doses of Compound A | | | | | | | |
| | Compound A Dose (uM) | | | | | | | |
| Model | 0 | 0.0024 | 0.01 | 0.039 | 0.2 | 0.6 | 2.5 | 10 |
| SW837 | 8.52 | 6.44 | 5.75 | 7.54 | 9.56 | 10.64 | 10.94 | 13.37 |
| T069C | 6.66 | 7.69 | 8.68 | 9.57 | 11.99 | 14.23 | 14.46 | 14.56 |
| NCI-H2122 | 10.22 | 7.91 | 9.2 | 11.7 | 15.37 | 17.27 | 18.08 | 18.47 |
| NCI-H2030 | 9.26 | 8.12 | 8.30 | 7.37 | 9.78 | 11.42 | 14.92 | 20.95 |
| BMS-2353 | 6.96 | 10.06 | 10.36 | 13.21 | 15.25 | 17.39 | 16.12 | 17.64 |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of treating cancer in a subject in need thereof, the method comprising administering to the subject (i) Compound A: 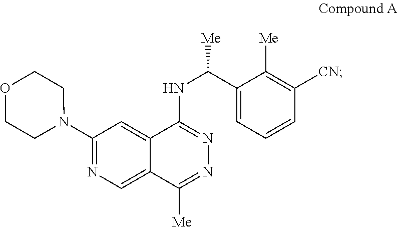 [SMILES: Cc1c(C#N)cccc1[C@@H](C)Nc1nnc(C)c2cnc(N3CCOCC3)cc12]
or a pharmaceutically acceptable salt thereof; (ii) a KRAS G12C inhibitor, and (iii) an EGFR inhibitor.

2. The method of claim 1, wherein the KRAS G12C inhibitor is adagrasib, or a pharmaceutically acceptable salt thereof.

3. The method of claim 1, wherein the EGFR inhibitor gefitinib, or a pharmaceutically acceptable salt thereof.

4. The method of any one of claim 1, wherein the cancer is pancreatic cancer, colorectal cancer, lung cancer, gastric cancer, breast cancer, bladder cancer, cervical cancer, ovarian cancer, cancer of the uterus, or a combination thereof.

5. The method of any one of claim 1, wherein the cancer is pancreatic cancer, colorectal cancer, or non-small cell lung cancer.
